(19) United States
(12) Patent Application Publication
(10) Pub. No.: US 2020/0330455 A1
(43) Pub. Date: Oct. 22, 2020

(54) METHOD FOR TREATING NEONATAL OPIOD WITHDRAWAL SYNDROME

(71) Applicant: CHIESI FARMACEUTICI S.P.A., Parma (IT)

(72) Inventors: Massimo CELLA, Parma (IT); Merran Ann THOMSON, Harrow on the Hill (GB); Walter Karl KRAFT, Philadelphia, PA (US)

(73) Assignee: CHIESI FARMACEUTICI S.P.A., Parma (IT)

(21) Appl. No.: 16/851,273

(22) Filed: Apr. 17, 2020

Related U.S. Application Data
(60) Provisional application No. 62/835,711, filed on Apr. 18, 2019.

(51) Int. Cl. A61K 31/485; A61K 47/38; A61K 9/00; A61K 9/08; A61P 25/36

(52) U.S. Cl. CPC A61K 31/485; A61K 47/38; A61P 25/36; A61K 9/08; A61K 9/006

(57) ABSTRACT

Formulations comprising buprenorphine are useful for treating opioid withdrawal syndrome.

### CROSS REFERENCES TO RELATED APPLICATIONS

This application claims priority to U.S. Provisional Patent Application No. 62/835,711, filed on Apr. 18, 2019, which is incorporated herein by reference in its entirety.

### BACKGROUND OF THE INVENTION

### Field of the Invention

The present invention relates to the treatment of neonatal abstinence syndrome. In particular, the present invention relates to methods for treating infants with neonatal opioid withdrawal syndrome.

### Discussion of the Background

Neonatal abstinence syndrome (NAS) is a complex of signs and symptoms in the postnatal period associated with the sudden withdrawal of maternally transferred opioids. The main manifestations include increased muscle tone, autonomic instability, irritability, poor sucking reflex, and impaired weight increase.

Neonatal opioid withdrawal syndrome (NOWS) is a subset of neonatal abstinence syndrome (NAS) and refers to neonatal withdrawal from opioid drugs and can occur in the presence of other drug withdrawal syndromes. NOWS is a generalized multisystem disorder that predominantly involves the central and autonomic nervous systems and the gastrointestinal tract. In the United States, neonatal withdrawal following in-utero opioid exposure is typically the result of prolonged maternal use and/or abuse of illicit or prescribed opioids during pregnancy. Birth leads to the abrupt cessation of fetal substance exposure and can precipitate acute withdrawal symptoms that can result in severe health complications with prolonged recovery and longer hospitalization. Withdrawal can be both severe and intense, with an estimated 60% to 80% of exposed neonates requiring pharmacological intervention to control symptoms (see Kraft, Walter K.; Stover, Megan W.; Davis, Jonathan M. Neonatal abstinence syndrome: Pharmacologic strategies for the mother and infant Seminars in Perinatology 40.3 (Apr. 1, 2016): 203-212, and Tolia V N, Patrick S W, Bennett M M, et al. Increasing incidence of the neonatal abstinence syndrome in U.S. neonatal ICUs. N Engl J Med. 2015; 372(22): 2118-2126, which are incorporated herein by reference in their entireties).

While the signs and symptoms of NOWS are similar to those experienced by adults undergoing acute opiate withdrawal, they present a higher risk to the neonate due to the infant's dependence on others for all aspects of well-being.

NOWS presenting signs include central nervous system hyperirritability (tremors, jitteriness, irritability, hyperactive muscle reflexes, and excessive high-pitched cry), autonomic nervous system deregulation and instability (tachypnea, nasal flaring, hyperphagia, temperature instability, insomnia, sweating, mottle skin, yawning, and sneezing), and gastrointestinal symptoms (diarrhea, vomiting, and poor feeding) (see Hudak M L, Tan R C. The Committee on Drugs and the Committee on Fetus and Newborn. Neonatal Drug Withdrawal. Pediatrics. 2012; 129; e540, and Kocheriakota P. Neonatal Abstinence Syndrome. Pediatrics. 2014; 134(2): e547-561, which are incorporated herein by reference in their entireties). Seizures are generally rare, although they have been reported occurring in 2% to 11% of neonate cases in the early stage of severe opioid withdrawal (see Doberczak T M, Kandall S R, Wilets I. Neonatal opiate abstinence syndrome in term and preterm infants. J Pediatr 1991; 118: 933-7, whivh is incorporated herein by reference in its entirety, especially if medical treatment has been delayed. A recent large observational cohort study of Medicaid babies in 46 US States reported an incidence of seizures was 2.7% among the 1705 observed cases of NAS (see Desai, R. J., Hernandez-Diaz, S., Bateman, B. T. & Huybrechts, K. F. Increase in prescription opioid use during pregnancy among Medicaid-enrolled women. Obstet. Gynecol. 123, 997-1002 (2014), which is incorporated herein by reference in its entirety. Neonates with NOWS can also experience weight loss or failure to thrive, which often results from a combination of poor feeding, vomiting, nausea, and diarrhea (see Kocheriakota P. Neonatal Abstinence Syndrome. Pediatrics. 2014; 134(2): e547-561, which is incorporated herein by reference in its entirety). If left untreated, some cases of NOWS may even lead to death (Sutter 2014). Some of the less severe signs and symptoms of opiate withdrawal may persist for several months (see Hudak ML, Tan RC. The Committee on Drugs and the Committee on Fetus and Newborn. Neonatal Drug Withdrawal. Pediatrics. 2012;129; e540, which is incorporated herein by reference in its entirety)

The onset, duration, and the severity of NOWS are dependent on several factors: the type of opiate exposure, maternal-fetal drug transferability, poly-drug exposure, time and duration of opiate exposure, placental and fetal drug concentrations, the rate of excretion, spatial representation and density of opiate receptors, and gestational age (see Hudak M L, Tan R C. The Committee on Drugs and the Committee on Fetus and Newborn. Neonatal Drug Withdrawal. Pediatrics. 2012; 129; e540, and Kraft, Walter K.; Stover, Megan W.; Davis, Jonathan M. Neonatal abstinence syndrome: Pharmacologic strategies for the mother and infant Seminars in Perinatology 40.3 (Apr 1, 2016): 203-212, which are incorporated herein by reference in their entireties).

The management of neonates at risk of this withdrawal syndrome is based on the following:

Identification of neonates at risk, preferably by identification of maternal substance exposure during pregnancy;
Early initiation of non-pharmacological care (this first-line treatment provides a suitable, supportive environment for both the mother and baby, which minimizes exposure to stress, and encourages breastfeeding where appropriate);

Monitoring for symptoms and signs of withdrawal; and

Pharmacological care, which is instituted when the symptoms and signs of withdrawal become too severe to be managed with non-pharmacological care. See Patrick S W, Schumacher R E, Horbar J D, et al. Improving Care for Neonatal Abstinence Syndrome. Pediatrics. 2016; 137(5):e20153835, which is incorporated herein by reference in its entirety.

There are no FDA-approved drugs for the treatment of NOWS with or without other concomitant drug withdrawal syndromes; however, there is consensus within the neonatology community that opioid replacement should be the first-line pharmacotherapy treatment (see Hudak M L, Tan R C. The Committee on Drugs and the Committee on Fetus and Newborn. Neonatal Drug Withdrawal. Pediatrics. 2012; 129; e540, Kocheriakota P. Neonatal Abstinence Syndrome. Pediatrics. 2014; 134(2): e547-561, and Jansson L M, Velez M, Harrow C. The opioid-exposed newborn: assessment and pharmacologic management. J Opioid Manag 2009; 5(1):47-55, which are incorporated herein by reference in their entireties). Morphine is the most often used opioid for the treatment of neonatal withdrawal following in-utero opioid exposure, followed by methadone, with some hospitals using buprenorphine. While morphine is used in more than 80% of NAS cases requiring pharmacological treatment (see Patrick S W, Schumacher R E, Horbar M, et al. Improving Care for Neonatal Abstinence Syndrome. Pediatrics. 2016; 137(5):e20153835, which is incorporated herein by reference in its entirety), there is no agreed standard of care or treatment regimens (see Kraft, Walter K.; Stover, Megan W.; Davis, Jonathan M. Neonatal abstinence syndrome: Pharmacologic strategies for the mother and infant Seminars in Perinatology 40.3 (Apr. 1, 2016): 203-212, which is incorporated herein by reference in its entirety) and the optimal pharmacological treatment protocols have not been established in large, well-controlled studies.

Commercially available liquid formulations of morphine and methadone are unsuitable for the neonatal population for several reasons. They contain inappropriately high concentrations of opioid, necessitating local compounding of extemporaneous formulations or dilution prior to administration within the hospital pharmacy or by neonatal nursing staff, increasing the risk of medication errors. Commercially available liquid formulations frequently contain alcohol and other preservatives and excipients that are contraindicated or undesirable in the neonatal population. Whilst an alcohol-free morphine solution is available, commercial methadone solutions contain alcohol. Therefore, the currently available pharmacological treatments for NOWS leave the neonate at risk of medication errors, other hazards related to local compounding of extemporaneous formulations, and exposure to alcohol, other preservatives, and excipients that are contraindicated or undesirable in the neonatal population.

Buprenorphine has several characteristics that make it an attractive agent in the treatment of NOWS. Buprenorphine is a long acting μ-opioid receptor partial agonist and κ-opioid receptor antagonist. The long half-life and duration of action prevents the rapid change in receptor occupancy that can precipitate withdrawal and should allow a simplified dosing regimen to be developed. Sublingual buprenorphine shows a lack of the cardiovascular liability that is associated with methadone. Related to the lack of cardiovascular liability, sublingual buprenorphine possesses a well-established safety profile in adults. Moreover, buprenorphine has the lowest incidence of respiratory depression among opioids, which is dose-dependent and has a ceiling effect in relation to the long half-life of buprenorphine (see Elkader A, Sproule B. Buprenorphine: clinical pharmacokinetics in the treatment of opioid dependence. Clin Pharmacokinet. 2005; 44(7):661-80, Walsh S L, Preston K L, Stitzer M L, et al. Clinical Pharmacology of buprenorphine: ceiling effects at high doses. Clin Pharmacol Ther. 1994; 55:569-80, Ohtani M. Basic pharmacology of buprenorphine. Eur J Pain Suppl. 2007; 1:69-73, Heel R C, Brodgern R N, Speight T M, Avery G S. Buprenorphine: a review of its pharmacological properties and therapeutic efficacy. Drugs. 1979; 17(2):81-110.dependent rat pups. B S Stoller et al.; Pediatric Anesthesia; 2004 14: 642-649, which are incorporated herein by reference in their entireties). However, buprenorphine is available as an ethanol-containing solution.

Furthermore, besides avoiding ethanol, it would be advantageous to provide a new therapeutic regimen for administering buprenorphine with the aim of simplifying it, while favoring the compliance of the neonate population.

In view of the above considerations, there is still a need to develop a safer and effective method for treating neonatal abstinence syndrome, in particular NOWS.

### SUMMARY OF THE INVENTION

Accordingly, it is one object of the present invention to provide novel methods for the treatment of opioid abstinence syndrome, in particular neonatal opiate abstinence syndrome.

It is another object of the present invention to provided novel methods for treating neonatal opioid withdrawal syndrome (NOWS).

These and other objects, which will become apparent during the following detailed description, have been achieved by the inventors' discovery of the formulations and methods described below.

Thus, in a first aspect, the present invention is directed to a method for treating opioid abstinence syndrome by administering to a patient in need thereof an effective amount of a propellant-free formulation, in form of aqueous solution, comprising of buprenorphine or a pharmaceutically acceptable salt thereof which is substantially free of ethanol.

In a second aspect, the present invention is directed to a method for treating opioid abstinence syndrome by administering to a patient in need thereof an effective amount of a propellant-free formulation, in form of aqueous solution, comprising of buprenorphine or a pharmaceutically acceptable salt thereof, which is substantially free of ethanol, for a period of 1 to 90 days, preferably of 3 to 70 days.

In a preferred embodiment, the formulation is administered sublingually. In another preferred embodiment, the formulation is a ready-to-use propellant-free pharmaceutical formulation in form of aqueous solution for sublingual and/or buccal administration, said formulation comprising:

(1) from 0.005 to 0.02% w/v of buprenorphine or a pharmaceutically acceptable salt thereof as the sole active ingredient;

(2) from 0.6% to 10% w/v of a thickening agent; and

(3) a buffering agent in an amount to provide a pH of 5.0 to 7.0. Its viscosity should be comprised between 500 and 2300 mPas, preferably between 700 and 2100 mPas at 25±2° C.

In a preferred embodiment of the invention, the thickening agent is a cellulose derivative, more preferably hydroxyethylcellulose (HEC) or sodium carboxymethylcellulose (NaCMC), even more preferably hydroxyethylcellulose.

In another aspect, the present invention is directed to the above ready-to-use pharmaceutical formulation in the manufacture of a medicament for the treatment of opioid abstinence syndrome, preferably neonatal abstinence syndrome, more preferably neonatal opioid withdrawal syndrome (NOWS).

Another aspect of the present invention refers to a method of treating of opioid abstinence syndrome in a patient in need thereof by administering the ready-to-use formulation of the invention for a period of 1 to 90 days, preferably of 3 to 70 days.

Preferably, the patient is a neonate affected by neonatal opioid abstinence syndrome, particularly NOWS.

The therapeutic method of the invention allows to obtain a quicker stabilization of the symptoms, as measured by the modified Finnegan score), a decreased use of adjunctive drugs and a shorter overall length of the treatment compared to the empirically-derived regimen of the prior art.

Furthermore, besides avoiding the use of ethanol, the therapeutic method of the invention turns out to be simpler than the regimen of the prior art, favoring the compliance of the neonate population.

### DETAILED DESCRIPTION OF THE PREFERRED EMBODIMENTS

With reference to buprenorphine, the terms “drug,” “active ingredient,” and “active substance” are used interchangeably.

The terms “neonates,” “newborns,” and “infants” are used interchangeably.

The terms “opioid abstinence syndrome” and “opiate withdrawal syndrome” are used interchangeably.

The term “safe” means a pharmaceutical formulation suitable for sublingual administration, well tolerated by neonates, and devoid of excipients that could be harmful, antigenic or toxic for said patient population.

The term “buccal and/or sublingual administration” encompasses the mucosal regions, i.e. the pharmacological route of administration by which substances diffuse into the blood through tissues under the tongue through the oral mucosa (tissues which line the mouth), being the most commonly used for systemic drug delivery. The buccal/sublingual routes bypass the GI tract and hence drugs absorbed in this way bypass the liver and first pass metabolism and have direct access to the systemic circulation.

The term “pH microenvironment” refers to the pH of mouth region of the patient immediately surrounding the formulation.

For a ready-to-use formulation, the expression “physically stable” refers to a formulation that, under long-term conditions (25° C.±2° C., 60%±2% relative humidity), exhibits substantially no precipitation of the active ingredient and/or excipient during storage for at least one month.

Due to analytical difficulties related to the determination of an active ingredient at low concentration, the expression “chemically stable” refers to a formulation that, upon storage, shows a change in the buprenorphine content of not more than ±15% and no drug related degradation products in an amount higher than 5% upon storage for at least one month.

The term “bioequivalent” means obtaining 80% to 125% of the Cmax and AUC values for a given active ingredient in a different product.

The term “therapeutically effective amount” means the amount of the active ingredient, that, when delivered to neonates, provides the desired biological effect.

The term “treatment” refers to the therapeutic use for palliative, curing, symptom-allievating, symptom-reducing, disease regression-inducing therapy.

The term “essentially consisting of” is used to indicate a formulation comprising only a thickening agent and a buffering agent as essential excipients. For example it could comprise a sweetener and/or flavoring agent, but not excipients such as permeation enhancers.

The term “weaning” refers to a procedure in place in the hospitals consisting of a gradual daily reduction in the opioids to avoid significant withdrawal discomfort.

With the term “Finnegan assessment score” is meant the Finnegan Neonatal Abstinence Scoring Tool (FNAST), developed by Loretta Finnegan, to assist health care providers in the assessment of withdrawal in the opiate-exposed infant. This tool contains a list of 21 withdrawal signs that are assessed and scored throughout the infant's withdrawal period (Finnegan L P, Connaughton J F Jr, Kron R E, and Emich J P Addict Dis 1975, 2(1-2), 141-148).

The Finnegan assessment score may be performed manually or through a mobile application, also referred to as an app, i.e. through a computer program or software application designed to run on a mobile device such as a phone, tablet, or other electronic tool.

Due to its activity on the opioid receptors, buprenorphine can successfully be used for the treatment of abstinence syndrome, particularly of neonatal abstinence syndrome.

Therefore, the aim of the present invention is to provide a safe pharmaceutical formulation for an efficacious treatment by sublingual and/or buccal administration to patients affected by opiate withdrawal syndrome (OWS), preferably neonates affected by neonatal OWS (hereinafter NOWS).

Said safe formulation shall comprise buprenorphine dissolved in a propellant-free aqueous vehicle.

The formulations of the present invention may be administered to neonates as well as to children or adolescents.

The formulation of the present invention can be in form of a dry powder to be dissolved extemporaneously before use or in form of ready-to-use formulation. In case it is dispensed as dry powder to be re-dissolved, it may be prepared according to known methods and it may be provided as a kit comprising a) the powdery pharmaceutical formulation; b) a pharmaceutically acceptable aqueous vehicle; c) a syringe; d) container means for containing the pharmaceutical formulation, the aqueous vehicle, and the syringe.

A ready-to-use formulation is preferably used.

Buprenorphine shall be utilized as a base or in the form of a pharmaceutically acceptable salt formed with an inorganic or organic acid, such as hydrochloric acid, hydrobromic acid, sulfuric acid, phosphoric acid, nitric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methanesulfonic acid, benzenesulfonic acid and toluenesulfonic acid.

Preferably, buprenorphine is present as the hydrochloride salt.

Advantageously, the concentration of the active ingredient shall be from 0.005 to 0.02% w/v, preferably 0.006 to 0.01% w/v, expressed as free base, based on the volume of the formulation.

In a particular embodiment of the invention, the concentration of buprenorphine hydrochloride is 0.0075% w/v, expressed as free base, based on the volume of the formulation.

The concentration of the thickening agent shall be comprised between 0.6% and 10%, w/v, preferably between 0.8%and 8.0% w/v, based on the volume of the formulation. The type and amount of the thickening agent shall be properly selected to achieve an adequate viscosity to retain the formulation as much as possible under the tongue of the patient, to minimize the absorption through the gastrointestinal tract. At the same time, the viscosity should be not too high to retard the release of the active ingredient from the matrix and hence, its local absorption.

More preferably, the concentration of the thickening agent could be between 1.0 and 6.0% w/v, based on the volume of the formulation.

In particular embodiments, said concentration is 1.0% w/v, or 1.5% w/v, or 2.0% w/v, or 6.0% w/v, based on the volume of the formulation.

Advantageously, the thickening agent may be selected from water-soluble polysaccharides such as alginates, carrageenans, pectin, water-soluble derivatives of cellulose: alkylcelluloses hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinyl alcohols, polyvinylacetatephthalates (PVAP), polyvinylpyrrolidone (PVP) and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired.

Preferably, the thickening agent is a water-soluble cellulose derivative selected from group consisting of hydroxyethylcellulose (HEC) or an alkali metal salt of carboxymethylcellulose (CMC) such as the sodium salt.

In fact, the thickening agents belonging to said classes could provide the suitable viscosity, while with other agents of the class of gums, such as xanthan gum, the viscosity of the formulation turned out to be too high.

Advantageously, the viscosity of the formulation at 25±2° C. shall be from 500 to 2300 mPas (1 mPas corresponds to 1 centipoise), preferably from 700 to 2100 mPas. The viscosity may be determined by any known method, for example using a rheometer.

Advantageously, the pH of the formulation of the present invention shall be from 5.0 to 7.0, more advantageously from 5.2 to 6.8, preferably from 5.5 to 6.5.

It has indeed been found that at pH higher than 7.0 the formulations of the invention may exhibit a lower chemical and physical stability.

Contrary to what reported in EP 2 461 795, which is incorporated herein by reference in its entirety, it has been found that said pH interval, avoiding the precipitation of the active ingredient into saliva, favors its absorption.

Without being limited by the theory, this may be due because no shift occurs with respect to the pH microenvironment (saliva/mucosa interface).

The results of a muco-adhesion test have also demonstrated that the formulations of the present invention are endowed with optimal property in terms of viscosity to allow its retention under the tongue of the patient without retarding the release of the active ingredient from the matrix, in particular when a water-soluble cellulose derivative is used as thickening agent.

Furthermore, according to in-vitro permeation experiments, when a water-soluble cellulose derivative is used, preferred formulations according to the invention shall have a pH of from 5.5 to 6.5 and an amount of thickening agent of from 1.0% to 2.0% w/v, even more preferably of 1.5% w/v, based on the volume of the formulation. The preferred thickening agent of this class shall be such as hydroxyethylcellulose. Said excipient is commercially available as Natrosol 250 HX™.

The in-vitro permeation tests simulate the passage of the drug across the oral mucosa and allow determination of its speed of release.

Any buffering agent able of providing the afore-mentioned pH maybe used, for example phosphate or citrate buffers as sodium or potassium salts. The skilled person in the art shall determine the proper amount.

In a preferred embodiment of the present invention, anhydrous citric acid and sodium citrate anhydrous is used as buffering agent.

The formulation of the present invention may also contain other excipients such as flavoring agents and/or sweeteners.

Flavoring agents may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors.

Other useful flavorings include aldehydes and esters such as benzaldehyde (cherry, almond), citral i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Stevia rebaudiana (Stevioside); chloro to derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof. Other sweeteners may also be used.

Typically, the skilled person in art shall select the sweetener and/or flavoring agent among those considered safe for neonatal administration.

Although not preferred, the formulations according to the invention may also contain permeation enhancers such as propylene glycol, and polyoxyl hydrogenated castor oil derivatives, for example polyoxyl 40 hydrogenated castor oil (commercially available as Kolliphor RH 40™).

In preferred embodiment of the present invention, the formulation has the following composition: 0.05 to 0.01% w/v buprenorphine hydrochloride expressed as a base, based on the volume of the formulation, 1.5% w/v hydroxyethylcellulose, based on the volume of the formulation, 0.12% w/v anhydrous citric acid, based on the volume of the formulation, 1.13% w/v sodium citrate anhydrous, based on the volume of the formulation, and water for injection. Its pH shall be of 6.0±0.3.

In an alternative preferred embodiment of the present invention, the formulation may have the following composition: 0.05 to 0.01% w/v buprenorphine hydrochloride expressed as a base, based on the volume of the formulation, 6.0% w/v sodium carboxymethylcellulose, based on the volume of the formulation, 0.12% w/v anhydrous citric acid, based on the volume of the formulation, 1.13% w/v sodium citrate anhydrous, based on the volume of the formulation, and water for injection. Its pH shall be of 6.0±0.3.

It has indeed been found, in an animal model of sublingual and/or buccal administration, that the aqueous formulations having the aforementioned compositions turned out to be bioequivalent to the alcoholic formulation disclosed in Kraft et al., Pediatrics 2008, 122(3), e601-607, which is incorporated herein by reference in its entirety.

Without being limited by the theory, this appears rather surprising, as ethanol would normally favor the absorption.

Therefore, according to a preferred embodiment of the present invention, the formulation shall only consist of a pharmaceutically acceptable salt of buprenorphine as the sole active ingredient, a thickening agent, a buffering agent in a proper amount to provide a pH of 5 to 7.0, and optionally a flavoring agent and/or a sweetener.

In particular, the formulation used in the present method is substantially free of ethanol. By substantially free of ethanol, it is meant that the formulation contains less than 1 wt. % ethanol, preferably less than 0.5 wt. % ethanol, more preferably less than 0.1 wt. % ethanol, even more preferably less than 0.01 wt. % ethanol, even more preferably no ethanol.

The formulation according to the present invention can be prepared according to known methods.

In a particular embodiment, the formulations of the invention are prepared according to the following steps:

(i) dissolving a suitable amount of buprenorphine or a pharmaceutically acceptable salt thereof, preferably its hydrochloride salt, in water in a proper container to obtain a concentrated clear solution;

(ii) optionally, sterilizing the obtained solution in step (i) by filtration;

(iii) in parallel, dissolving the appropriate amount of a buffering agent in water in a suitable volumetric container until a clear solution is obtained;

(iv) optionally, sterilizing the obtained solution in step (iii) by heating;

(v) adding the appropriate volume of buprenorphine concentrated solution to the buffering agent solution to obtain the final desired concentration of buprenorphine, preferably 0.05-0.01 mg/mL (as free base) while mixing under continuous stirring; and

(vi) adding slowly the proper amount of the thickening agent to the solution of step (v) under continuous stirring until the thickening agent is completely dissolved and a clear, homogenous solution is obtained.

Preferably, water is water for injection (WFI).

In step (i) the concentration shall vary from 0.1 to 0.5 mg/ml, preferably from 0.2 to 0.4 mg/ml, more preferably of 0.324 mg/mL.

Advantageously, the container in steps (i) and (iii) could be made of any suitable material such as plastic or glass.

The sterilization procedures of step (ii) and (iv) shall be carried out according to methods known in the art.

In particular, the porosity of the filter in step (ii) and the temperature of heating in step (iv) shall be suitably adjusted by the skilled person.

The final formulation shall be distributed, under aseptic conditions, in the suitable containers.

Advantageously, the formulation according to the present invention may be used for the treatment of neonatal abstinence syndrome of any severity.

Preferably, the formulation of the invention may be used for the treatment of patients affected by opioid withdrawal syndrome (OWS) with or without exposure to other drugs, more preferably for the treatment of neonates affected by NOWS.

Typically, the buprenorphine dosage may vary between 10 and 110 micrograms/kg birth body weight per day, preferably between 30 and 90 micrograms/kg birth body weight per day, and, and it could be administered divided in two or more doses, preferably three doses for a period ranging from 1 to 90 days, preferably 3 to 70 days.

The dose and the duration of the treatment shall be anyway adjusted by the physician, depending on the weight of the neonate and the severity of the neonatal abstinence syndrome.

Optionally, if used for the treatment of NOWS, the formulation is poured under the tongue by a syringe followed by insertion of a pacifier in the mouth of the neonate to reduce swallowing.

Any syringe available on the market of a volume comprised between 0.1 to 2.5 ml, more advantageously between 0.5 and 2.0 ml, could be used.

Syringes of 0.5 ml or 1.0 ml could preferably be used.

The syringe may be made of plastic, glass or any suitable material, preferably of plastic, more preferably of cyclo-olefin polymer (COP).

For example, syringes from Becton Dickinson (New Jersey, USA) could be suitable.

In a preferred embodiment of the invention, the ready-to-use formulation might be provided as pre-filled in a syringe, preferably in COP, and without a Luer lock.

For instance, suitable pre-fillable syringes are commercially available from Gerresheimer AG (Dusseldorf, Germany).

When they are used with neonates, said pre-fillable syringe shall be without a needle and endowed with a suitable cap, preferably big enough to avoid chocking of the neonate in case of its accidental ingestion.

In an alternative embodiment, a package comprising the pharmaceutical formulation of the invention in form of either ready-to-use aqueous solution or powder to be reconstituted in a suitable aqueous vehicle, in combination with a suitable syringe may be provided.

Typically, neonates with with a gestational age (GA) ≥36 weeks shall be treated, preferably having and the sum of 3 consecutive Finnegan assessment scores ≥24. A definition of the Finnegan score is also reported in Kraft KW et al., New Engl J Med, 2017, 376 (24)-2341-2348, which is incorporated herein by reference in its entirety.

A symptoms score for neonatal abstinence syndrome could also be established as reported in Chervoneva I et al J Perinatology https://doi.org/10.1038/s41372-020-0606-4, published on line on Feb. 20, 2020, which is incorporated herein by reference in its entirety.

According to the therapeutic method of the invention, neonates shall receive a sublingual dose of the buprenorphine formulation at a starting dose of 10 microg/kg every 8 hours.

Other parameters, such as duration of the treatment shall adjusted by the physician on the basis the severity of the symptoms, birth weight, sex etc.

According to a preferred posology scheme, initial doses are higher versus the prior art (8 to 12 microg/kg, preferably 10 microg/kg vs. 5 to 6 microg/kg) with a quicker up-titration. Dose increments are based on fixed constant amounts (4 to 6 micro/kg, preferably 5 microg/kg) rather than percentages (25%) of the previous dose. Weaning is preferably quicker (15% vs. 10%). Finally, once the dose reverts back to the same value as the starting dose, the frequency of administration moves from every 8 hours to every 12 hours, and then every 24 hours. In a particularly preferred dosage regime, the initial dose is 8 to 12 microg/kg, preferably 10 microg/kg, three times per day, for a total daily dose of 24 to 36 microg/kg, preferably 30 microg/kg. If the Finnegan assessment score is from 18 to 24, then the initial dose is maintained. If the Finnegan assessment scores is above 24, then the dose is increased by an increment of 4 to 6 microg/kg, preferably 5 microg/kg, up to a dose of 26 to 34 microg/kg, preferably 30 microg/kg, three times per day, for a total daily dose of 78 to 102 microg/kg, preferably 90 microg/kg. If the Finnegan assessment score is below 18, then weaning is started. In the above amounts, microg/kg represents microg per kg of body weight at birth.

Other features of the invention will become apparent in the course of the following descriptions of exemplary embodiments which are given for illustration of the invention and are not intended to be limiting thereof.

### EXAMPLES

### Example 1

### Formulation 1

The following formulation was prepared:

| Formulation 1 pH = 6.0 | Quantity per mL (mg) | Function |
|---|---|---|
| Buprenorphine HCl | 0.075* | Active Principle |
| | | Ingredient |
| Anhydrous Citric Acid | 1.22 | Buffering Agent |
| Sodium Citrate Anhydrous | 11.3 | Buffering Agent |
| Hydroxyethylcellulose | 15.0 | Thickening Agent |
| Water for Injection | 100 ml | Solvent |

*expressed as a base.

### Example 2

### The Treatment of NOWS

In a randomized, multicenter, double blind, double-dummy, parallel group, controlled study, 99 babies with neonatal opioid-withdrawal syndrome (NOWS) with or without other concomitant drug withdrawal syndromes are randomized to receive the formulation of Example 1 or morphine treatment plus the corresponding matched placebo.

Pharmacological treatment starts up to 7 days after delivery in babies who show signs of NOWS and have failed to respond to non-pharmacologic care. Withdrawal signs are be assessed using a predefined modified Finnegan neonatal abstinence assessment tool. Assessment is recorded every 4 hours (±1 hour).

Following a randomization list, babies are assigned to one of the two arms:
Test arm: babies receive a sublingual dose of the formulation of Example 1 at a starting dose of 10 μg/kg every 8 hours (adjusted according to the birth weight).
Reference arm: babies receive an oral dose of morphine at a starting dose of 0.07 mg/kg every 4 hours (adjusted according to the birth weight).

Pharmacological treatment consists of the following phases; initiation, escalation, stabilization, weaning and cessation. Assessment of the need for treatment and dose adjustments is based upon clinical signs of withdrawal evaluated using a Finnegan Neonatal Abstinence Scoring Tool (FNAST) and continued until at least 48 hours after the last dose of opioid treatment. A review of the drug dose will take place on a daily basis or more frequently during the escalation phase to ensure timely dose adjustment.

The duration of follow up is for 6 weeks and 48 hrs after the final opioid treatment dose. Evidence of recurrence of significant withdrawal is monitored for all babies who remain within the hospital. For babies discharged following the required period of in patient observation (48 hours post last opioid treatment dose), daily telephone contact with the primary caregiver (parental/legal guardian or foster mother) continues for the first 7 days and records the infant's wellbeing and identify any escalation of withdrawal signs.

Thereafter weekly telephone contact continues for the duration of the follow up period. Significant escalation of withdrawal signs warrants clinical review and assessment for relapse severe enough to require pharmacological treatment and readmission.

The end of the trial is defined as the last follow-up at 6 weeks contact of the last subject in the trial.

A 18-month (±1 months) follow-up visit to assess neurodevelopmental and general health status and confirmation of the safety of the test treatment is undertaken by the participating centers. This assessment will be evaluated separately from the main part of the study.

### Subject Selection Criteria

### Subject Recruitment

99 neonates with neonatal opioid-withdrawal syndrome (NOWS) with or without other concomitant drug withdrawal syndromes are randomized.

### Inclusion Criteria

Neonates must meet all of the following inclusion criteria to be eligible for enrolment into the study:
1. Written informed consent obtained by parents/legal representative (according to local regulation) prior to or after birth.
2. Birth weight ≥3^{rd} centile for gestational age (GA), according to the CDC growth chart
3. Gestational age ≥36 weeks
4. Exposure to opioids during the last month of fetal life
5. Signs of neonatal opioid withdrawal syndrome requiring treatment, and the sum of 3 consecutive Finnegan assessment scores is ≥24 or a single score ≥12

### Exclusion Criteria

The presence of any of the following excludes a subject from study enrolment:
1. Familiar history of prolonged QTc syndrome
2. Major congenital malformations or evidence of congenital infection
3. Signs of fetal alcohol spectrum disorders
4. Maternal alcohol abuse, defined as average of 3 or more drinks per week in the last 30 days
5. Medical illness at the time of randomization, including but not exclusively:
   a) Neonatal hypoglycaemia requiring intravenous glucose therapy
   b) Neonatal respiratory illness requiring non-invasive or invasive respiratory support
   c) Hypoxic ischemic encephalopathy and seizures
   d) Severe hyperbilirubinemia-bilirubin at or above the exchange transfusion threshold as defined by the AAP
   e) Severe elevation of serum aminotransferases
   f) Proven or suspected early onset neonatal infection which will require more than 48 hours treatment with antibiotics
6. Unable to tolerate an oral or sublingual medication
7. Need for medications forbidden in this study protocol
8. Any condition that, in the opinion of the Investigator, would place the neonate at undue risk
9. Participation in another clinical trial of any placebo, experimental medical device or biological substance conducted under the provisions of a protocol on the same therapeutic target. The participation in studies involving diagnostic devices or treatments for conditions other than NOWS and NAS may be permitted following an agreement with the Sponsor. Non-interventional observational studies are allowed

### Subject Withdrawal

Babies are discontinued from the study for any of the following reasons:
An adverse event occurs that, in the opinion of the investigator, makes it unsafe for the subject to continue in the study. In this case, the appropriate measures will be taken.
Additional drug therapy is required when withdrawal signs as assessed by the FNAST are not controlled with the maximum dose of the study drug in combination with phenobarbital.
Prolonged requirement for opioid therapy defined as >10 weeks (>70 days) i.e. inability to wean from opioid therapy. In this situation it is likely that the baby's withdrawal is compounded by in utero polysubstance exposure.
The baby is lost to follow-up.
Parents or legal guardian withdraws consent.
The baby's safety is affected by violation of inclusion or exclusion criteria or use of not-permitted concomitant medication.
The baby transfers to another site
The sponsor or the regulatory authorities or the Ethics Committee(s), for any reason, terminates the entire study, or terminates the study for this trial site or this particular subject.

### Treatment(s)

The medications include:

### The Formulation of Example 1:

liquid preparation 0.075 mg/mL (Test treatment), sublingual administration

Active ingredient: Buprenorphine

Excipients: Citric buffer pH 6, Hydroxyethylcellulose

Presentation: transparent solution

Placebo, the Formulation of Example 1 without Buprenorphine:

sublingual administration

Excipients: Citric buffer pH 6, Hydroxyethylcellulose

Presentation: transparent solution

Morphine Sulfate Injection 0.5 mg/ml Vial (Reference Treatment)

Active ingredient: morphine sulfate

Excipients: water USP

Presentation: clear solution

Sterile water for injection used as Morphine matched placebo

Sterile water for Injection: USP is a sterile, nonpyrogenic preparation of water for injection which contains no bacteriostatic, antimicrobial agent or added buffer and is supplied only in single-dose containers. Water for Injection, USP is chemically designated H2O.

### Dosage and Administration

### Starting Dose

The starting dose for the formulation of Example 1 is 10 μg of buprenorphine/kg of body weight (30 μg/kg/day).

Frequency of Administration

The formulation of examples 1 is administered every 8 hours, together with oral placebo for morphine. Oral placebo for morphine is administered also every 4 hours, to match the morphine administration schedule.

There is a ±1 hour interval around each nominal time point to account for sleeping and feeding schedule.

If dosing occurs at a time different from the specified nominal time, the next dose is scheduled to take place 8 hours following the actual dose administration.

Lost doses: if a baby spits or vomits soon after administration, the administration is not repeated in order to avoid potential over medication.

Dose Escalation

In the up-titration phase, dose escalation will occur if the sum of the 3 consecutive Finnegan assessment scores is ≥24 OR a single score is ≥12 OR a rescue dose was administered.

No more than one dose escalation can take place each day unless a rescue dose is required.

During the up-titration phase, doses will increase by 5 ug of buprenorphine/kg of body weight steps, i.e. after the initial 10 ug/kg dose there can be consecutive dose increases to 15, 20, 25 and 30 ug/kg, if signs of withdrawal persist. The maximum dose (90 ug/kg/day) is therefore reached, if needed, after 4 up-titration steps.

### Weaning

The initiation of weaning begins after stabilization, i.e. when the signs of NOWS are controlled (as defined by a period of 48 hours without further drug treatment escalation). Doses are decreased by 15% once per day if the sum of the previous 3 scores is <18 and no single score is >8.

If the sum of the previous three scores is ≥28 and at the discretion of the treating physician, the standing dose reverts to the previous dose or dose interval at which symptoms were controlled.

Dose Cessation

The cessation dose is around the initial dose, (i.e. between 90-110% of the 10 ug/kg initial dose). Once this is reached, the dosing frequency is decreased from q8 to q12 hours for 24 hours, followed by a further decrease in dosing frequency from q12 to q24 hours prior to stopping.

Following cessation of dosing, babies are observed in an inpatient setting for at least 48 hours, during which time scoring of NOWS symptoms will continue.

### Reference Treatment Dosage

Starting Dose

The starting dose for morphine is 0.07 mg of morphine/kg of body weight (0.42 mg/kg/day).

Frequency of Administration

Morphine is administered every 4 hours.

Sublingual placebo for the formulation of Example 1 will be administered every 8 hours, to match the formulation of Example 1 administration schedule. There is a ±1 hour interval around each nominal time point to account for sleeping and feeding schedule. If dosing occurs at a time different from the specified nominal time, the next dose is scheduled to take place 4 hours following the actual dose administration.

Lost doses: if a baby spits or vomits soon after administration, the administration will not be repeated in order to avoid potential over medication.

Dose Escalation

In the up-titration phase, dose escalation occurs if the sum of the 3 consecutive Finnegan assessment scores is ≥24 OR a single score is ≥12 OR a rescue dose was administered.

No more than one dose escalation takes place each day unless a rescue dose is required.

During the up-titration phase, after the initial 0.07 mg of morphine/kg of body weight dose there can be consecutive dose increases to 0.09, 0.12, 0.16 and 0.21 mg/kg, if signs of withdrawal persist.

The maximum dose (1.25 mg/kg/day) is therefore reached, if needed, after 4 up-titration steps.

### Weaning

The initiation of weaning begins after stabilization, i.e. when the signs of NOWS are controlled (as defined by a period of 48 hours without further drug treatment escalation). Doses are decreased by 15% once per day if the sum of the previous 3 scores is <18 and no single score is >8.

If the sum of the previous three scores is ≥28 and at the discretion of the treating physician, the standing dose reverts to the previous dose at which symptoms were controlled.

### Dose Cessation

The cessation dose will be at 0.025 mg/kg q 4 hours.

Following cessation of dosing, babies are observed in an inpatient setting for at least 48 hours, during which time scoring of NOWS symptoms will continue.

### Rescue Dose

If, between scheduled doses, a baby has a single score ≥12, a rescue dose of the formulation of Example 1 or morphine is administered at the discretion of the treating physician.

The rescue dose will be same as the previous dose.

A rescue dose is given at least 1 hour after the previous dose and 1 hour before the next scheduled dose.

The rescue dose is not given once the maximum dose has been reached.

The administration of a rescue dose in the up-titration period triggers a dose escalation at the next schedule dose.

The administration of a rescue dose in the weaning period does not trigger a dose escalation.

If after cessation of therapy a rescue dose is given, the baby is observed for 24 hours.

To maintain the blinding, both active drug as well as placebo will be administered at the same time.

### Randomised Treatment Period:

Test treatment: Formulation of Example 1 Arm

Formulation of Example 1, 0.075 mg of buprenorphine per mL administration every 8 hours

Morphine Matched Placebo

The maximum does of the Formulation of Example 1 dose is 90 μg buprenorphine per kilogram of body weight per day

### Reference Treatment: Morphine Arm

Morphine solution 0.5 mg/ml administration every 4 hours

Formulation of Example 1 matched placebo

The maximum morphine dose is 1.25 mg per kilogram of body weight per day.

### Administration

The frequency of administration is 8 hours for test treatment and of 4 hours for reference treatment, therefore the morphine placebo of morphine is administered every 4 hours in the formulation of Example 1 arm and the placebo will be administered every 8 hours in the comparator arm.

To allow for alteration in sleeping and feeding schedules, each dose of drug is administered ±1 hour around nominal time point for that dose.

Administration of oral morphine/morphine placebo will be according to standard of care.

### Study Schedule

Potential mothers are identified through the outpatient treatment clinics. All babies from whom consent has been obtained will have NOWS graded according to FNAST. Pharmacological treatment starts up to 7 days after delivery in babies who have persisting signs of NOWS and despite the implementation of appropriate non-pharmacologic care. Babies with a GA ≥36 weeks and with the sum of 3 consecutive Finnegan assessment scores ≥24 or a single score ≥12 are eligible for randomization.

The study is conducted as follows for each baby:
A pre-screening visit on the mothers to confirm the used opioid in the last month of pregnancy and to verify the eligibility of the neonate for inclusion in the study. This visit will be carried out during the last month of pregnancy or after delivery.
A screening visit on the babies who show sign of NOWS. The visit will be carried out up to 7 days after delivery.
A randomization visit to assign the babies to a treatment and provide first treatment administration.
Treatment period: from randomization up to 48 hours after the last dose. The treatment period can last up to 10 weeks (70 days) from the first treatment dose.
Regular follow up will continue both before and after discharge from hospital to identify those babies who suffer recurrence of significant withdrawal. The duration of follow up will be for 6 weeks and 48 hrs after the final opioid treatment dose. Evidence of recurrence of significant withdrawal will be monitored for all babies who remain within the hospital. For babies discharged following the required period of in-patient observation (48 hours post last opioid treatment dose), daily telephone contact with the primary caregiver (parental/legal guardian or foster mother) will continue for the first 7 days, and will record the infant's wellbeing and identify any escalation of withdrawal signs. Thereafter weekly telephone contact will continue for the duration of the follow up period.

A 18-months (±1 months) follow-up visit for a further long-term assessment and confirmation of the safety of the test treatment.

### Pre-Screening Visit

A pre-screening visit is carried out in order to identify mothers with history of opioid use in the last month of pregnancy.

### Screening Visit

After delivery a screening visit is carried out up to 7 days of age on the babies with signs of NOWS in order to identify eligible babies for the study.

The following information will be collected:
NOWS score: a predefined modified Finnegan neonatal abstinence assessment tool (FNAST) will be used to assess withdrawal signs every 4 hours (±1 hour)
Gestational age, sex
Race/ethnicity
Birth weight, head circumference
Baby urine toxicology data (optional)
Vital signs: heart rate (HR), respiratory rate (RR), peripheral oxygen saturation (SpO₂), body temperature (BT). Blood pressure (DBP; MBP; SBP) will be recorded if collected according site clinical practice
Hematology and blood chemistry data: the results of investigations preformed prior to randomization will be recorded; full blood count (FBC), urea, creatinine and electrolytes (sodium, potassium, magnesium calcium, phosphorus), glucose, C-reactive protein if collected before randomization according to clinical practice
Intended feeding option: breastfeeding or formula feeding
Medical illness
Inclusion/exclusion criteria
AEs and concomitant medication to be check and recorded. Non-serious AEs will not be collected for screening failure.

### From Randomization to Stabilization

Once the baby's eligibility to take part in the study is confirmed, the Investigator will randomize the baby to the assigned treatment by using the IRT system. Pharmacological treatment starts up to 7 days after delivery in babies who show signs of NOWS and that have failed to respond to non-pharmacologic care.

The following information will take place:
NOWS score: a predefined modified Finnegan neonatal abstinence scoring tool (FNAST) will be used to assess withdrawal signs to check and confirm eligibility. Signs of withdrawal will be assessed and recorded every 4 hours (±1 hour) Randomization: Babies with a GA ≥36 weeks and the sum of 3 consecutive Finnegan assessment scores ≥24 or a single score ≥12 will be assigned in a 2:1 ratio to either the formulation of Example 1 or morphine. Treatment doses will be calculated according to birth weight (Time 0).
Vital signs: heart rate (HR), respiratory rate (RR), peripheral oxygen saturation (SpO₂), and body temperature (BT) will be collected at the same time of the NOWS score, except for BT which will be collected at least once per day. Blood pressure (DBP; MBP; SBP) will be recorded if collected according site clinical practice.
Treatment administration: treatment doses will be calculated according to birth weight and the NOWS score which will be assessed and recorded every 4 hours

Feeding status: breastfeeding, formula feeding, mixed feeding, and the proportion of maternal breastmilk intake during this period of time i.e. randomization to stabilization.
Liver function test (AST, ALT): after treatment stabilization, prior to the first weaning dose
Blood sampling for the formulation of Example 1 pharmacokinetics. Further concomitant medications and adverse events as well as changes in those already reported.

### From Weaning to the End of Treatment

The following information is collected:
NOWS score: signs of withdrawal will be assessed and recorded every 4 hours (±1 hour) using FNAST
Vital signs: heart rate (HR), respiratory rate (RR), peripheral oxygen saturation to (SpO₂), body temperature (BT) will be collected at the same time of the NOWS score but only once a day. Blood pressure (DBP; MBP; SBP) will be recorded if collected according site clinical practice
Treatment administration: treatment doses will be calculated according to birth weight and the NOWS score which will be assessed and recorded every 4 hours.
Liver function test (AST, ALT) 48 hours after last treatment dose
Feeding status: breastfeeding, formula feeding, mixed feeding, and the proportion of maternal breastmilk intake during this period of time i.e. weaning to end of treatment
Blood sampling for formulation of Example 1 pharmacokinetics.
Further concomitant medications and adverse events as well as changes in those already reported.

### End of Treatment Period Visit

The following procedures take place:
NOWS score
Vital signs: heart rate (HR), respiratory rate (RR), peripheral oxygen saturation (SpO₂), body temperature (BT), blood pressure (DBP; MBP; SBP) will be recorded if collected according site clinical practice
Neurological and behavioural assessment
Head circumference, and body weight and length
Feeding status: breastfeeding, formula feeding, mixed feeding, and the proportion of maternal breastmilk intake during this period of time i.e. end of treatment to discharge.
Further concomitant medications and adverse events as well as changes in those already reported.

### Follow-Up

Regular follow up will continue both before and after discharge from the hospital to identify those babies who suffer recurrence of significant withdrawal.
During the first 7 days after 48 hours from the last dose:
   a) If the baby has already been discharged from the hospital, daily telephone contact with the primary caregiver (parental/legal guardian or foster mother). The following information will be recorded:
      General wellbeing
      Escalation of withdrawal signs
      Feeding status: breastfeeding, formula feeding or mixed feeding
      Further concomitant medications and adverse events as well as changes in those already reported.
   b) If the baby stays in the hospital, the following information will be recorded:
      General wellbeing
      Escalation of withdrawal signs
      Feeding status: breastfeeding, formula feeding, mixed feeding, and the proportion of maternal breastmilk intake during this period of time i.e. first 7 days.
      Further concomitant medications and adverse events as well as changes in those already reported.

Once the baby has been discharged, weekly telephone contact continue until 6 weeks +48 hours from the last dose. The following information will be recorded
General wellbeing

Escalation of withdrawal signs
Feeding status: breastfeeding, formula feeding or mixed feeding
Further concomitant medications and adverse events as well as changes in those already reported.

### Follow-Up: 18 Months (±1 Months).

A further clinical assessment at 18 months (±1 months) is performed by physicians of a multidisciplinary team at the participating study centres to assess the general wellbeing, growth and long-term neurodevelopmental assessment and growth parameters. This 18-month clinical assessment is analysed and evaluated separately from the initial part of the study and be object of an addendum to the initial core clinical study report.

Regular contact continues after the initial 6 weeks and 48 hour period to in order to maintain contact with the baby and family until the planned formal assessment of neurodevelopmental and general health status at a further clinical assessment will be performed.

The 18-month assessment of general wellbeing, growth, and development use a range of measurements, tests and questionnaires including the Bayley Scales of Infant Development (BSDI) III.

### Investigations

### NOWS Score

The Finnegan Neonatal Abstinence Scoring System Tool (FNAST) is used to assess the severity of NOWS.

### Feeding Status

Feeding status is collected daily from the electronic patient record. (Formula /breast milk or a mixed feeding and proportion of maternal breast milk intake).

### Toxicological Urine Collection

Is performed in those hospitals where is common clinical practice, according to site procedures.

### Vital Signs

At screening and from the randomization until the end of the treatment, heart rate (HR), respiratory rate (RR), peripheral oxygen saturation (SpO₂), body Temperature (BT) will be collected. Blood pressure (DBP; MBP; SBP) are recorded if collected according site clinical practice.

From randomization up to the stabilization they are collected at the same time of the NOWS score, except for BT which will be collected at least once per day.

From weaning until the end of the treatment they are collected once a day at the time of NOWS score.

### Hematology/Blood Chemistry and Liver Function

The results of investigations if preformed according to site clinical practice prior to randomization are recorded; full blood count (FBC), urea, creatinine and electrolytes (sodium, potassium, magnesium calcium, phosphorus), glucose, C-reactive protein,

The baseline for hematology and chemistry will be collected before randomization according to clinical practice.

Liver-function test analysis (aspartate aminotransferase [AST], alanine aminotransferase [ALT]),) are performed on 3 occasions: after the first dose, at stabilization and 48 hours after the last treatment dose.

### Pharmacokinetics

Blood sampling for PK occurs the day of randomization and then every 5±1 days until end of treatment. The first blood sampling is taken after the first dose. Subsequent sampling should occur after the morning dose and be timed to fit in with the following sampling windows; one blood sample should be taken in the 0-2 h post-dose time window, one in the 2-4 h post-dose time window and one in the 4-8 h post-dose time window.

If possible, in order to minimize discomfort for the babies, PK samples are collected simultaneously to routine blood sample collection.

Blood concentrations are determined using validated HPLC-MS/MS methods. The laboratory analysis is carried out following Good Laboratory Practice (GLP) regulations of the OECD. The analytical procedure will be described in a separate analytical study plan.

### Neurological and Behavioral Assessment

Neurological and behavioral assessment are performed at the end of treatment period.

### Efficacy Assessments

### Primary Efficacy Endpoint

Duration of treatment defined as the number of hours from start of the treatment until the last dose of study drug.

### Secondary Efficacy Endpoints:

Time to first weaning, defined as the number of hours from start of the treatment until the first dose reduction
Use of adjunctive drug therapy (phenobarbital) for symptoms of NOWS
Total hours of treatment with adjunctive therapy
Use of rescue doses (Formulation of Example 1 or morphine)
Number of rescue doses administered
Percentage of total dose which is from rescue doses
Length of opioid related hospital stay, defined as number of days from day of birth until 48 hours after the final dose of drug treatment for NOWS
Relapse of NOWS, defined as experiencing recurrence of significant signs of withdrawal
Incidence of readmissions, defined as readmission to hospital for NOWS relapse

### Safety Assessments

### During Administration of Sublingual Formulation of Example 1 or Oral Morphine:

Number and percentage of babies with peri-dosing adverse events (AEs) (i.e., mouth irritation or inflammation, apnoea, desaturation, brady/tachycardia, cough, immediate swallowing of sublingual drug, regurgitation, vomiting occurring after administration)

### During the Study

From the randomization to the end of the treatment the following information is collected and presented using summary statistics:
Adverse events and adverse drug reactions (ADRs).
Liver enzymes analysis-function testing (AST, ALT,) will be performed on 3 occasions: after the first dose, at stabilization and after the last treatment dose.
Vital signs: heart rate (HR), respiratory rate (RR), peripheral oxygen saturation (SpO2), body temperature (BT) will be monitored, and data collected during the escalation and stabilization phases.
Body Weight, head circumference,
Hematology and blood chemistry: full blood count (FBC), urea, creatinine and electrolytes (sodium, potassium, magnesium calcium, phosphorus), glucose, C-reactive protein if collected before randomization according to site clinical practice.
Blood pressure (SBP, MBP, DBP) will be monitored according to individual site clinical practice

### End of the Treatment Period

After 48 hours from the last dose the following is recorded and presented using summary statistics:
Vital signs: heart rate (HR), respiratory rate (RR), peripheral oxygen saturation (SpO₂), body temperature (BT). Blood pressure (SBP. MBP. DBP) if collected according to site clinical practice
Neurological and behavioral assessment
Body weight, length and head circumference
All the Adverse Events, and adverse drug reactions, and concomitant medications as well as changes in those already reported.

For ethical, practical and organizational reasons, the clinical study will be performed under double blind conditions until the last dose of buprenorphine formulation is administered, then a functional unblinding will occur.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for treating opioid abstinence syndrome, comprising administering to a patient in need thereof an effective amount of a propellant-free pharmaceutical formulation in form of an aqueous solution, comprising buprenorphine or a pharmaceutically acceptable salt thereof, wherein said formulation is substantially free of ethanol, for a period of 1 to 90 days.

2. The method according to claim 1, wherein said formulation comprises:
(1) from 0.005 to 0.02% w/v, based on the volume of the formulation, of buprenorphine or a pharmaceutically acceptable salt thereof as the sole active ingredient;
(2) from 0.6 to 10% w/v, based on the volume of the formulation, of a thickening agent; and
(3) a buffering agent in an amount sufficient to provide a pH of 5.0 to 7.0.

3. The method according to claim 1, wherein said buprenorphine is in the form of its hydrochloride salt.

4. The method according to claim 1, wherein said formulation has a pH of from 5 2 to 6.8.

5. The method according to claim 1, wherein said formulation has a pH of from

5. 5 to 6.5.

6. The method according to claim 1, wherein said formulation has a viscosity of from 500 to 2300 mPas at 25±2° C.

7. The method according to claim 1, wherein said formulation has a viscosity of from 700 to 2100 mPas at 25±2° C.

8. The method according to claim 2, wherein said thickening agent is a cellulose derivative.

9. The method according to claim 2, wherein said thickening agent is hydroxyethylcellulose or sodium carboxymethylcellulose.

10. The method according to claim 2, wherein said buffering agent is made of citric acid and sodium citrate.

11. The method according to claim 1, wherein said formulation further comprises a sweetener and/or a flavoring agent.

12. A method according to claim 1, wherein said formulation consists essentially of:
05-0.01% w/v, based on the volume of the formulation, of buprenorphine hydrochloride expressed as a base;
1.5% w/v, based on the volume of the formulation, of hydroxyethylcellulose or 6.0% w/v, based on the volume of the formulation, of sodium carboxymethylcellulose;
0.12% w/v, based on the volume of the formulation, of anhydrous citric acid;
1.13% w/v, based on the volume of the formulation, of sodium citrate anhydrous; and
water for injection.

13. The method according to claim 1, wherein said formulation is administered sublingually and/or buccally.

14. The method according to claim 1, wherein said syndrome is neonatal opioid abstinence syndrome.

15. The method according to claim 14, wherein said syndrome is neonatal opioid withdrawal syndrome (NOWS).

16. The method according to claim 14, wherein said buprenorphine is administered in an initial dose of 8 to 12 microgram/kg of body weight at birth, three times per day, for a total daily dose of 24 to 36 microgram/kg of body weight at birth, and if the Finnegan assessment score of said infant is from 18 to 24, then the initial dose is maintained, if the Finnegan assessment scores of said infant is above 24, then the dose of said buprenorphine is increased by an increment of 4 to 6 microgram/kg of body weight at birth, up to a maximum dose of 26 to 34 microgram/kg of body weight at birth, to be administered three times per day, for a total daily dose of 78 to 102 microgram/kg of body weight at birth, and if the Finnegan assessment score of said infant is below 18, then weaning is started.

17. The method according to claim 16, wherein said buprenorphine is administered in an initial dose of about 10 microgram/kg of body weight at birth, three times per day, for a total daily dose of about 30 microgram/kg of body weight at birth, and if the Finnegan assessment score of said infant is from 18 to 24, then the initial dose is maintained, if the Finnegan assessment scores of said infant is above 24, then the dose of said buprenorphine is increased by an increment of about 5 microgram/kg of body weight at birth, up to a maximum dose of about 30 microgram/kg of body weight at birth, to be administered three times per day, for a total daily dose of about 90 microgram/kg of body weight at birth, and if the Finnegan assessment score of said infant is below 18, then weaning is started.
